(19) United States
(12) Patent Application Publication
(10) Pub. No.: US 2006/0178389 A1
(43) Pub. Date: Aug. 10, 2006

(54) Use of siramesine in the treatment of cancer

(76) Inventors: Christian Thomsen, Stroby (DK); Marcel Leist, Valby (DK); Marja Jaattela, Copenhagen (DK); Marie Stampe Ostenfeld, Copenhagen (DK)

Correspondence Address: LUNDBECK RESEARCH USA, INC.;ATTENTION: STEPHEN G. KALINCHAK, LEGAL, 215 COLLEGE ROAD, PARAMUS, NJ, 07652, US

(21) Appl. No.: 11/314,796

(22) Filed: Dec. 21, 2005

Related U.S. Application Data
(63) Continuation of application No. PCT/DK04/00885, filed on Dec. 17, 2004.

(30) Foreign Application Priority Data
Dec. 19, 2003 (DK) PA200301889

(51) Int. Cl. A61K 31/4747

(52) U.S. Cl. 514/278

(57) ABSTRACT

The present invention relates to the treatment of cancer. In particular, the invention provides pharmaceutical compositions comprising siramesine for the treatment of cancer. The invention further provides a method of treatment comprising administering siramesine to a patient suffering from cancer.

### FIELD OF INVENTION

The present invention relates to the use of Siramesine for the preparation of medicaments useful for the treatment of cancer.

### BACKGROUND OF THE INVENTION

Tumor cells have often acquired resistance towards classical treatment modalities, such as classical caspase-mediated apoptosis. However, there is still a large unmet need for novel efficient drugs for the treatment of cancers.

It is known that sigma2 receptors are upregulated in cells representing many different types of cancers (Bem et al. 1991, Cancer Res. 51, 6558; Vilner et al. 1995, Cancer Res. 55, 408), and furthermore that sigma2 receptor ligands may inhibit cell proliferation and induce apoptosis in tumor cells (Brent & Pang, 1995, Eur. J. Pharmacol. 278, 151; Crawford & Bowen, 2002, Cancer Res. 62, 313). Additionally, it has been shown that sigma 2 ligands may potentiate the activity of antineoplastic drugs (Crawford & Bowen, 2002, Cancer Res. 62, 313).

International Patent Publication No. WO 92/22554 describes a series of sigma receptor ligands considered useful for the treatment of a range of psychic and neurological disorders. The structure activity relationship of these compounds has been further investigated by Perregaard, J. et al., J. Med. Chem., 1995, 38, 11, p. 1998-2008.

Among numerous other compounds WO 92/22554 discloses the compound 1′-[4-[1-(4-fluorophenyl)-1H-indole-3-yl]-1-butyl]-spiro[isobenzofuran-1(3H)4′-piperidine]

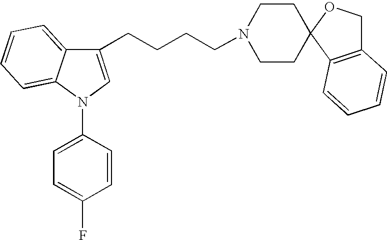

(Siramesine), the novel use of which is the subject of the present invention.

We have now, surprisingly found that Siramesine is significantly more potent in its anti-carcinogenic activities, than reference sigma2 ligands.

### DESCRIPTION OF THE INVENTION

According to the present invention a medicament for the treatment of cancer is provided.

Sigma2 receptor ligands have been known to induce apoptosis in cancer cells of different origin. We have now surprisingly found that Siramesine of the invention, when used alone is more potent in inducing apoptosis in cancer cells than sigma2 active reference compounds such as haloperidol. Furthermore, we have observed a significant synergistic effect of Siramesine when used in combination with known chemotherapeutic compounds such as etoposide, doxorubicin, staurosporin, vincristine and tamoxifen. The present invention therefore demonstrate that Siramesine may be used for the manufacture of pharmaceutical compositions for the treatment of cancer, and that such compositions may be used in combination with other chemotherapeutic cancer drugs, and/or in combination with radiotherapy. In the combined use of Siramesine and anticancer chemotherapeutic drugs, the drugs may be administered simultaneously or sequentially.

In one embodiment, the present invention relates to the use of Siramesine or a pharmaceutically acceptable salt thereof, together with a chemotherapeutic drug in a synergistic effective dose for the preparation of a pharmaceutical composition as above, which is adapted for simultaneous administration of the active ingredients. In particular, such pharmaceutical compositions may contain the active ingredients within the same unit dosage form, e.g. in the same tablet or capsule. Such unit dosage forms may contain the active ingredients as a homogenous mixture or in separate compartments of the unit dosage form.

In another embodiment, the present invention relates to the use of Siramesine or a pharmaceutically acceptable salt thereof together with a chemotherapeutic drug in a synergistic effective dose for the preparation of a pharmaceutical composition or kit as above, which is adapted for sequential administration of the active ingredients. In particular, such pharmaceutical compositions may contain the active ingredients in discrete unit dosage forms, e.g. discrete tablets or capsules containing either of the active ingredients. These discrete unit dosage forms may be contained in the same container or package, e.g. a blister pack.

As used herein the term kit means a pharmaceutical composition containing each of the active ingredients, but in discrete unit dosage forms.

As used herein, the term “synergistic effective dosage” means the dosages of Siramesine and the chemotherapeutic agent at which their combined use provides a synergistic effect, preferably the maximal obtainable synergistic effect.

The pharmaceutical composition or kit of the invention may be adapted for simultaneous administration of the active ingredients or for sequential administration of the active ingredients, as described above.

More specifically, the present invention relates to the novel use of Siramesine having the general formula

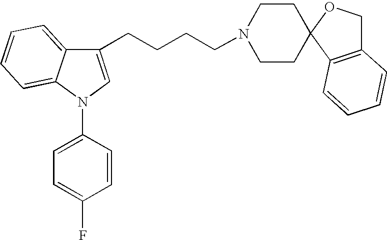

or pharmaceutically acceptable salts thereof for the preparation of a medicament for the treatment of cancer.

Moreover, the present invention relates to a method for the treatment of cancer comprising administering to an individual in need thereof a pharmaceutically acceptable amount of Siramesine or a pharmaceutically acceptable salt thereof.

In a further aspect the invention relates to a method for treatment of cancer comprising administering Siramesine or a pharmaceutically acceptable salt thereof to an individual to be treated with or undergoing treatment with a chemotherapeutic agent.

According to the invention the compound 1′-[4-[1-(4-fluorophenyl)-1H-indole-3-yl]-1-butyl]-spiro[isobenzo-furan-1(3H),4′-piperidine] (Siramesine) may be used as the base of the compound or as a pharmaceutically acceptable acid addition salt thereof or as an anhydrate or hydrate of such salt. The salts of the compound used in the invention are salts formed with non-toxic organic or inorganic acids. Exemplary of such organic salts are those with maleic, fumaric, benzoic, ascorbic, succinic, oxalic, bis-methylenesalicylic, methanesulfonic, ethane-disulfonic, acetic, propionic, tartaric, salicylic, citric, gluconic, lactic, malic, mandelic, cinnamic, citraconic, aspartic, stearic, palmitic, itaconic, glycolic, p-amino-benzoic, glutamic, benzene sulfonic and theophylline acetic acids, as well as the 8-halotheophyllines, for example 8-bromo-theophylline. Exemplary of such inorganic salts are those with hydrochloric, hydrobromic, sulfuric, sulfamic, phosphoric and nitric acids. Preferably the compound is used as the fumarate or the hydrochloric salt.

The fumarate of 1′-[4-[1-(4-fluorophenyl)-1H-indole-3-yl]-1-butyl]-spiro[isobenzofuran-1(3H),4′-piperidine] can be prepared as described in Perregaard, J. et al., J. Med. Chem., 1995, 38, 11, 1998-2008 (compound 14f) and the base and other pharmaceutically acceptable salts may be obtained there from by standard procedures.

Thus the acid addition salts according to the invention may be obtained by treatment of 1′-[4-[1-(4-fluorophenyl)-1H-indole-3-yl]-1-butyl]-spiro[isobenzo-furan-1(3H),4′-piperidine] with the acid in an inert solvent followed by precipitation, isolation and optionally re-crystallisation by known methods and if desired micronisation of the crystalline product by wet or dry milling or another convenient process, or preparation of particles from a solvent-emulsification process.

Precipitation of the salt is preferably carried out in an inert solvent, e.g. an inert polar solvent such as an alcohol (e.g. ethanol, 2-propanol and n-propanol).

According to the invention, 1′-[4-[1-(4-fluorophenyl)-1H-indole-3-yl]-1-butyl]-spiro[isobenzofuran-1(3H),4′-piperidine] or a pharmaceutically acceptable salt thereof may be administered in any suitable way e.g. orally or parenterally, and it may be presented in any suitable form for such administration, e.g. in the form of tablets, capsules, powders, syrups or solutions or dispersions for injection. Preferably, and in accordance with the purpose of the present invention, the compound of the invention is administered in the form of a solid pharmaceutical entity, suitably as a tablet or a capsule or in the form of a suspension, solution or dispersion for injection.

Methods for the preparation of solid pharmaceutical preparations are well known in the art. Tablets may thus be prepared by mixing the active ingredients with ordinary adjuvants and/or diluents and subsequently compressing the mixture in a convenient tabletting machine. Examples of adjuvants or diluents comprise: corn starch, lactose, talcum, magnesium stearate, gelatine, lactose, gums, and the like. Any other adjuvant or additive such as colourings, aroma, preservatives, etc. may also be used provided that they are compatible with the active ingredients.

As used herein, the term “composition” is intended to encompass a product comprising the specified ingredients in the specific amounts, as well as any product which results, directly or indirectly, from combination of the specific ingredients in the specified amounts.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, microcrystalline cellulose, sodium crosscarmellose, corn starch, or alginic acid; binding agents, for example starch, gelatin, polyvinyl-pyrrolidone or acacia, and lubricating agents, for example, magnesium stearate, stearic acid or talc.

The compositions of the invention may be used for treatment of cancer in mammals, preferably in humans.

Siramesine or a salt thereof for use of the invention is most conveniently administered orally in unit dosage forms such as tablets or capsules, containing the active ingredient (calculated as the free form) in an amount from about 0.01 μg/kg/day to 100 mg/kg/day, preferably 0.01 μg/kg/day to 30 mg/kg/day body weight, most preferably 0.5 mg/day/kg to 7.0 mg/day/kg body weight.

When siramesine is combined with other compounds in order to obtain an increased effect, or in order to allow for the use of a subnormal dose of the other compound, to minimize side effects, then subnormal doses of siramesine and/or the other compound may be used for the treatment. Calculation of patient specific doses is routine practice for those skilled in the art.

The pharmaceutical compositions and methods provided in the present invention are particularly deemed useful for the treatment of cancer including solid tumors such as skin, breast, brain, cervical carcinomas, testicular carcinomas, etc.. More particularly, cancers that may be treated by the compounds, compositions and methods of the invention include, but are not limited to: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); Genitourinarv tract: kidney (adenocarcinoma, Wilm's tumor [nephroblastoma], lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple mycloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoina, melanoma), vagina (clear cell carcinoma, squamous cell carcinoma, botryoid sarcoma (embryonal rhabdomyosarcoma), fallopian tubes (carcinoma); Hematologic: blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma]; Skin: malignant melanoma, basal cell carcinoma, squamous cell carcinoma, Karposi's sarcoma, moles dysplastic nevi, lipoma, angioma, dermatofibroma, keloids, psoriasis; and Adrenal glands: neuroblastoma.

Thus, the term “cancerous cell” as provided herein, includes a cell afflicted by any one of the conditions identified above, and the term “cancer” includes but is not limited to any of the conditions identified above. Any of the above mentioned conditions are to be considered as single embodiments, and the compositions directed to the treatment of each condition may accordingly be claimed individually or be included in the claimed group when the term cancer is used.

Whenever any of the above cancer indications are mentioned in relation to use of siramesine, a pharmaceutical composition, a kit, a method of treatment it is intended to be an individual embodiment. Accordingly, each of the indications specified above may individually be claimed together with said use of siramesine, pharmaceutical composition, kit, method of treatment and method for the identification of compounds useful for treatment.

### EXPERIMENTAL PROCEDURE

Cell Culture

Murine fibrosarcoma cells WEHI-S, Wn902, Wn912, and WEHI-R4 are normal, vector control, Hsp70 overexpressing, and hTNF resistant cells, respectively. Other cell types tested include: Human breast cancer cell types MCF7-S1 and MDA-MB-468, and non-tumorigekic immortalized breast epithelial cells HBL100. MCF7 casp3.1 and −3.3 and neo2 are single cell clones expressing caspase3 and vector. MCF7 pCEP, -Bcl2 WT, -Bcl2 NT, -Bcl2 Acta, and -Bcl2 Cb5 are single cell clones expressing vector, wildtype Bcl2, cytoplasma localized Bcl2, mitochondria localized Bcl2, and ER localized Bcl2 (Maria Høyer Hansen, Apoptosis Laboratory, Danish Cancer Society). Human neuroblastoma cell line SK-N-MC cells (ATCC, USA). In addition human cervix carcinoma cell lines HeLa (kindly provided by Dr. J. Lukas, Danish Cancer Society) and ME180 were tested. HEK293-A, prostata cancer cell line PC3, and non-tumorigenic immortalized prostata epithelial cell line PNT1A were tested as well. Non-transformed N1H3T3 murine fibroblasts were kindly provided by C. Holmberg (University of Copenhagen, Denmark). Fibroblasts were transduced with pBabe-puro mock, -SV40LT, -v-Ha-ras, -c-src as described (Fehrenbacher et al, 2004). Cells were propagated in DMEM (Invitrogen, Paisly, UK) supplemented with 10% heat-inactivated calf serum (Biological Industries, Beit Haemek, Israel), 0.1 mM non-essential amino acids (Invitrogen), and antibiotics or RPMI-1640 (Invitrogen) supplemented with 6% heat-inactivated calf serum and antibiotics at 37° C. in a humidified air atmosphere with 5% CO₂. Cells were repeatedly tested and found negative for mycoplasma by hoecsht staining (H-33342, Molecular Probes, Eugene, Oreg.).

[³H]Siramesine Binding to Cell Membranes or Tissue Membranes

The presence of Siramesine-sensitive binding sites on tissue prepared from cell lines as indicated above or tissue from rodent or human were demonstrated using [³H]Lu 28-179 (Siramesine) binding assay described previously Søby, K. et al., Neuropharmacol., 2002, 43, 95-100. In brief, cells were cultured as described, harvested in phosphate buffered saline using a cell scraber and centrifuged (1000×g, 10 min). The resulting pellets were used for [3H]Lu 28-179 binding assays as described in Søby et al., 2002.

Apoptotic Stimuli

The following apoptotic stimuli were tested: Siramesine (Lu-28-179), Siramesine analogs: 28131M, 28134M, 292880, 32160F, 32124C, and 32168F, and haloperidol (H. Lundbeck A/S, Copenhagen, Denmark), human TNF-α (Strathmann Biotech Gmbh, Germany), thapsigargin, etoposide, doxorubicin, staurosporine, vincristine, tamoxifen (SIGMA-Aldrich, St Louis, Mo.), concanamycinA (Alexis Biochemicals, San Diego, Calif.).

Pharmacological Inhibitors and Drugs

The following protease inhibitors were used: zVAD-fmk, DEVD-fmk (Bachem Bubendorf, Switzerland), DEVD-CHO (Neosystems, Strasbourg, France), LEHD-CHO, zFA-fmk (Enzyme System Products, Livermore, Calif.), CA-074-Me (Peptides International, Louisville, Ky.), APC1138 (Celera Applied Biosystems, Foster City, Calif., USA),. Pepstatin A, PD150606, and calpain inhibitor I (CI) (Calbiochem, La Jolla, Calif.), TPCK (Boehringer Mannheim), pefabloc (AEBSF) (Roche Diagnostics, DK).

The following antioxidants were used: Butylated hydroxyanisole (BHA), α-tocopherol, γ-tocopherol, glutathione ethyl ester (GSH), N-acetyl-cysteine (NAC) (SIGMA-Aldrich, St Louis, Mo.). Cells were pre-incubated with inhibitors and antioxidants 1 h prior to drug.

In addition, the effect on siramesine cytotoxicity of the following drugs were tested: sigma2-receptor antagonists BD1047 (Tocris) and AC927 (gift of W. Bowen, Brown University, USA), 3-methyl-adenine, ActinomycinD, cyclohexamide and cholesterol.

Detection of Cell Death

Cell Viability

Cell viability was measured using the MTT reduction assay. Conversion of the tetrazolium salt 3-(4,5-dimethylthiazole-2-yl)-2,5-diphenyltetrasodiumbromide (MTT, SIGMA-Aldrich) into the blue-colored formazan product was assessed spectrophotometrically by absorption at 570 nm using a Versamax microplate reader (Molecular Devices). The survival rate was determined as the percentage of untreated cells or inhibitor-treated cells: Cells were seeded into 96-well plate, containing 200 μL media, and treated the following day with drugs. The effect on cell viability was assessed 24-60 h after drug addition by removal of 100 μL media and addition of 25 μL MTT solution (1 mg/mL in PBS, sterile filtered). After 3 h incubation 37° C. in darkness, the cells were permeabilized using 100 μL solubilization buffer (20% SDS in 50% dimethylformamide solution) and analyzed on spectrophotometer the following day.

Cell Death and Cytotoxicity

Cell death and cytotoxicity was estimated using the lactate dehydrogenase (LDH) release assay (Roche, Mannheim, Germany). Rupture of the plasma membrane releasing cytoplasmic LDH into the culture media is a measure of cytotoxicity or cell lysis. The enzymatic acticity of LDH resulting in oxidation of lactate to pyruvate and reduction of NAD⁺ to NADH+H⁺ was measured spectrophotometrically by the conversion of tetrazolium salt (yellow) to formazan salt (red). Cells were seeded and treated as described for MTT assay. Upon analysis, 50 μL media was removed and and mixed with 50 μL reaction buffer. The remaining media was removed and cells lysed in 1% Triton X-100 for 20 min at 37° C. The LDH content was measured in the cell lysate as well, and an estimate of % LDH release was calculated:
Cytotoxicity (% LDH release)=LDH_{in media}/(LDH_{in media}+LDH_{in lysate})×100%

Nuclear Condensation

The cell death mode was assessed by the type of nuclear condensation and performed by hoechst staining (cell permeable Hoechst 33342, SIGMA-Aldrich) of drug-treated cells. Non cell permeable Sytox Green (Molecular Probes) was used to assay loss of membrane integrity and compared with nuclear morphological changes observed with Hoechst. Cells were incubated with 10.000× dilution of 25 mg/mL hoechst and /or 10.000× dilution of 5 mM sytox green for 5 min at 37° C. where after type of nuclear condensation and possible co-stain was analyzed using an inverted Olympus microscope IX-70.

Gel electrophoresis for delection of DNA laddering. Cells were harvested and incubated at 50° C. overnight in 120 μL lysis buffer (100 mM NaCl, 100 mM Tris-HCl (pH 8.0), 25 mM EDTA, 0.5% SDS and 100 μg/mL proteinase K). The samples were precipitated with 6M NaCl and centrifuged at 13.000 rpm for 5 min at 4° C. Genomic DNA was subsequently precipitated from the supernatant by 2.5 volumes of 96% ethanol. After centrifugation at 20.000 rpm for 10 min at 4° C. and rinsing with 70% ethanol, the pellets were dissolved in 10 mM Tris-HCl (pH 8.0) and 1 mM EDTA containing 1 μg/mL Rnase A. The samples were incubated at 37° C. for 1.5 h, and the DNA concentration was estimated from absorbance (A) at 260 nm. DNA (5 μg/lane) was electrophorezed on 1.5% agarose gel and visualized by ethidium bromide staining.

Caspase and Cathepsin Activity

To measure cytosolic cystein cathepsin enzyme activity, subconfluent cells seeded in 24-well plates were treated with an extraction buffer (250 mM sucrose, 20 mM HEPES, 10 mM KCl, 1.5 mM MgCl₂, 1 mM EDTA, 1 mM EGTA, 1 mM pefablock; pH 7.5) containing 20 μg/mL digitonin for 12-15 min on ice. To measure total cellular cystein cathepsin enzyme activity, cells were treated with the above extraction buffer containing 200 μg/mL digitonin for 12-15 min on ice. For analysis of caspase 3/7-like activity subconfluent cells were treated with caspase extraction buffer (0.5% Triton X-100, 25 mM HEPES, 5 mM Mg₂Cl, 1 mM EGTA, 1 mM pefablock, pH 7.5) for 20 min on ice. The caspase 3/7-like activity and cystein cathepsin activities were estimated by adding one volume of 20 μM Ac-DEVD-AFC (Biomol) in caspase reaction buffer (100 mM HEPES, 20% glycerol, 0.5 mM EDTA, 0.1% CHAPS, 5 mM DTT, 1 mM pefablock, pH 7.5) or 20 μM zFR-AFC (Enzyme System Products) in cathepsin reaction buffer (50 mM sodium acetate, 4 mM EDTA, 8 mM DTT, 1 mM pefablock, pH 6.0), respectively. The Vₘₐₓ of the liberation of AFC (excitation 400 nm, emission 489 nm) was analyzed over 20 min at 30° C. using a Spectramax Gemini fluorometer (Molecular Devices, Sunnyvale, Calif.).

Lysosomal Stability Assay, Cell Culture

Cells were exposed to the lysomotropic weak base and metachromatic fluorochrome acridine orange (AO, Molecular Probes) that accumulates in acidic compartments. When highly concentrated in acidic lysosomes AO shows red fluorescence, but upon relocalozation green fluorescence in cytoplasma. In order to monitor lysosomal integrity, subconfluent drug-treated cells were exposed to 0.1-0.5 μg/mL AO for 3 h at 37° C. Cells were either evaluated using an inverted Olympus microscope IX-70 or detached from the substratum and analyzed by flow cytometry using a FACSort (Becton Dickinson, San Jose, Calif.) with an argon ion laser with an output wavelength of 488 nm and analyzed using CELLQuest software.

Lysosomal Stability in Vitro Assay

MCF-7 cells seeded in 14 cm plates were loaded with Fe-dextran for 9 h, followed by 16 h clearance in culture medium. Subsequently, the cells were washed in PBS and detached from the substratum. The cell pellet was resuspended in SCA buffer (20 mM Hepes KOH, 10 mM KCl, 1.5 mM Mg₂Cl, 1 mM EDTA, 1 mM EGTA, 250 mM sucrose, pH 7.5) and equilibrated on ice for 20 min. The cell pellet was homogenized by 150-200 strokes using a Teflon pestle. After centrifugation for 5 min at 750 g, the supernatant was isolated and the centrifugation step was repeated. The remaining supernatant was transferred to a column contained in a magnetic field. The lysosomal fraction was washed twice and eluted from the column. The release of lysosomal cathepsins was subsequently measured in 25 μL lysosome solution in black costar 96 well plates as described for cathepsin activity measurements. After 1.5 hour reaction at 37° C., the Vₘₐₓ of the liberation of AFC was analyzed over 20 min at 30° C. using a Spectramax Gemini fluorometer (Molecular Devices, Sunnyvale, Calif.).

Immunoblot Analysis and Immunofluorescence

The primary antibodies used included mouse monoclonal antibodies against cathepsin B (Oncogene Research Products, Boston, Mass.), cathepsin L (Transduction Laboratories, Lexington, Ky.), cytochrome c (BD Pharmingen), and glyceraldehyde-3-phosphate dehydrogenase (GAPDH, Biogenesis, Poole, UK), Hsp70 (2H9; kindly provided by Boris Margulis, St. Petersbrug, Russia) and rabbit polyclonal cathepsin D (DAKO corporation, CA), and AIF (apoptosis lab, Danish Cancer Research). For immunoblot analysis, proteins were separated by 6-12% SDS-PAGE and transferred to a nitrocellulose membrane. After primary antibody incubation 1 h at RT or 4° C. overnigh the blot was incubated with secondary horseradish-peroxidase-conjugated goat anti-mouse or -rabbit IgG antibody 1 h at RT. Subsequent protein detection was performed using ECL or ECL plus (Amersham Biosciences, Buckinghamshire, England).

To visualize cathepsins, AIF, and cyt C, cells were fixed and permeabilized in −20° C. methanol for 10 min at 25° C. After preblocking with 5% goat serum (in 1% BSA, 0.3% Triton X-100 in PBS) for 20 min, cells were incubated with primary antibodies (in 0.25% BSA, 0.1% Triton X-100 in PBS) as indicated for 1.5 h. After 1 h incubation with secondary antibodies Alexa Fluor-488 or -546-conjugated anti-mouse IgG or anti-rabbit IgG (Molecular Probes) cells were washed three times in 0.05% Tween 20 in PBS and mounted using ProLong Antifade Kit (Molecular Probes). Confocal analysis was conducted using a Zeiss Axiovert 100M microscope with LSM 510 software.

In Vivo Experiments

WEHI-R4 cells (5×10⁶) were implanted subcutaneously in the back of immunocompetent female BALB/c mice. Siramesine treatment commenced two days prior to tumor implant. Mice were divided into groups (5-9/group) and administered peroral 200 μL 1) vehicle (0.5% methylcellulose 15 in 0.9% NaCl solution), 2) 100 mg/kg/day Siramesine in suspension (in 0.5% methylcellulose 15 in 0.9% NaCl solution) 7 days per week. For combinational in vivo studies, 50 mg/kg siramesine was administered 7 days/week in combination with a single dose of etoposide (i.p.) up to 30 mg/kg (administered on day one). Tumor volumes were estimated by the use of a caliper. The effect of the drugs on the tumor growth was monitored over 14-21 days before the mice were sacrificed.

Results

In a dose dependent manner, Siramesine effectively induces programmed cell death in cultured tumor cell lines of various origins, including cell lines originating from tumors of the prostate, breast and cervix. The sensitivity of cells towards Siramesine is increased upon the oncogenic transformation by ras or src, indicating that siramesine has the desired ability of a cancer drug to induce its effects selectively on the cancer cells with only limited effects on normal cells.

Furthermore, when tested against reference sigma ligands such as Haloperidol and pentazocine in WEHI-S and MCF7 cells, siramesine was a significantly more potent inducer of apoptosis than Haloperidol and pentazocine was.

The mode of death induced by siramesine is caspase-independent based on the nuclear morphological changes during the death process, the absence of protection by pharmacological caspase inhibitors, and the absence of effector caspase activation Instead, release of lysosomal cathepsins into the cytosol seems to be involved in the execution of the death process. This finding is based on immmunohistochemical stainings of lysosomal cathepsin B and L as well as the in vitro release of cathepsins from purified lysosomes. Also, the use of pharmacological inhibitors of cathepsins can attenuate the death induced by siramesine.

Tumor cells that were protected against most other anti-cancer drugs by ectopic expression of Bcl-2 were effectively killed by siramesine.

Whereas some chemotherapeutics activate a p53-dependent death pathway, siramesine does not activate p53. This indicates that the death pathway differs significantly from that induced by DNA damaging agents (such as etoposide), a result that supports the data showing a broad cancer indication range for siramesine, since the p53-dependent death pathway is often compromised in cancer. This is further supported by the above mentioned observation that Siramesine induced tumor cell death is not inhibited by Bcl-2.

Importantly, siramesine was well tolerated in vivo and showed an anti-tumorigenic effect in a syngenic tumor xenograft model in BALB/c mice. In addition, a combinational effect of siramesine and etoposide was observed in vivo as compared to groups single-treated with siramesine and etoposide respectively. Furthermore, siramesine worked in a synergistic manner together with etoposide, doxorubicin, staurosporin, vincristine and tamoxifen in induction of cell death in WEHI-S cells. These results show that siramesine is a novel anti-cancer drug which is especially effective as compared to other reference sigma ligands, and which may be used alone or in combination with conventional chemotherapeutics for the treatment of cancer.

1. A method of treating a subject suffering from cancer comprising administering to the subject a therapeutically effective amount of Siramesine or a pharmaceutically acceptable salt thereof for the treatment of cancer.

2. A method of treating a subject comprising administering to the subject Siramesine or a pharmaceutically acceptable salt thereof in an amount effective to augment and/or provide an enhanced effect of a chemotherapeutic agent.

3-4. (canceled)

5. The method according to claim 1 wherein the cancer is selected from the group consisting of lung cancer, prostate cancer, breast cancer, glioma, neuroblastomas, melamoma, leukemia, bone marrow cancer and skin cancer.

6. The method according to claim 2 wherein the chemotherapeutic agent is selected from the group consisting of etoposide, doxorubicin, staurosporin, vincristine, and tamoxifen, or pharmaceutically acceptable salts thereof.

7. The method of claim 1 wherein said treatment of cancer is combined with radiotherapy.

8. The method of claim 1 wherein the fumarate or hydrochloride salts of Siramesine are administered.

9. (canceled)

10. A pharmaceutical composition comprising Siramesine or a pharmaceutically acceptable salt thereof and a chemotherapeutic agent.

11. A kit comprising Siramesine or a pharmaceutically acceptable salt thereof and a chemotherapeutic agent.

12-13. (canceled)

14. A method of treating a subject suffering from cancer comprising administering to the subject a therapeutically effective amount of Siramesine or a pharmaceutically acceptable salt thereof and a chemotherapeutic agent.

15. A method of treating a subject suffering from cancer comprising administering to the subject a therapeutically effective amount of Siramesine or a pharmaceutically acceptable salt thereof, a first chemotherapeutic agent and a second chemotherapeutic agent.

16. The method according to claim 14 wherein the chemotherapeutic agent is selected from the group consisting of etoposide, doxorubicin, staurosporin, vincristine, and tamoxifen, or pharmaceutically acceptable salts thereof.

17. The method according to claim 15 wherein the first chemotherapeutic agent is selected from the group consisting of etoposide, doxorubicin, staurosporin, vincristine, and tamoxifen, or pharmaceutically acceptable salts thereof.
